(19) United States
(12) Patent Application Publication
(10) Pub. No.: US 2026/0272606 A1
(43) Pub. Date: Sep. 17, 2026

(54) SURGICAL SPECIMEN MAPPING MECHANISM

(71) Applicant: Emet Surgical Inc., Aurora, CO (US)

(72) Inventor: Robert Witkow, Denver, CO (US)

(21) Appl. No.: 19/671,084

(22) Filed: May 7, 2026

Related U.S. Application Data
(63) Continuation-in-part of application No. 18/630,111, filed on Apr. 9, 2024.
(60) Provisional application No. 63/496,133, filed on Apr. 14, 2023.

(51) Int. Cl. A61B 90/00

(52) U.S. Cl. CPC A61B 90/39; A61B 2090/3908; A61B 2090/3962; A61B 2090/3991

(57) ABSTRACT

The herein disclosed invention constitutes a surgical specimen margin orientation marking mechanism comprising a plurality of surgical specimen margin markers configured to provide a mapping between a tumor mass and surrounding tissue, wherein the improvement comprises each surgical specimen margin marker comprising sutures placed on tissue to identify one or more locations at which tissue is to be removed or surrounding tissue.

### CROSS-REFERENCE TO RELATED APPLICATIONS

This application is a Continuation-in-Part of U.S. patent application Ser. No. 18/630,111, filed Apr. 9, 2024, which claims the benefit of priority of U.S. Provisional Patent Application No. 63/496,133, filed Apr. 14, 2023, the contents of which are all incorporated herein by reference in their entirety.

### FIELD OF THE INVENTION

The present invention relates to medical devices, more particularly, surgical specimen margin orientation marking.

### BACKGROUND OF THE INVENTION

Surgical specimen margin orientation marking for endoscopic, robotic, laparoscopic, or other surgery types, where body tissue is removed, is critical for pathological diagnosis, tumor excision and other margin marking. One example of surgical specimen margin orientation marking may be featured in an endoscopic excision of a tumor. In such an example, the tumor is removed and forwarded to a pathologist for evaluation by frozen or permanent section. The pathologist subsequently performs an analysis to identify malignancy at one or more margins. A surgeon may then direct additional tissue to be excised, the orientation of which is critical for proper and accurate excision.

The current technique for surgical specimen margin orientation marking involves ink marking after a specimen has been removed from a body. One embodiment of a conventional marking technique wherein a specimen mass is marked with a dotted line to differentiate from surrounding tissues identifies an excised mass. In such a technique an ink mark may be placed at “12:00 o'clock” position on the mass.

However, this approach is inaccurate and subject to error if the orientation of the specimen is changed (e.g., dropped, manipulated, etc.). For example, it may be difficult to find the ink marks and compare to those on the removed mass. Further, the surgeon, staff, or pathologist examining the mass may mistakenly manipulate the removed mass, or there may be miscommunication between surgeons and pathologists related to orientation. An alternative approach that may be implemented includes placement of a reference suture in the specimen at the 12:00 O'clock position. However, this approach is equally subject to error.

My prior U.S. Pat. No. 10,111,727 discloses the use of marking clips for specimen marking-one marking clip is placed on a mass, while the second marking clip is placed in the surrounding tissue. Color coded sutures are attached to the clips having colors designating the anterior, inferior, superior, posterior, and deep margins. There are certain surgical procedures in which the placement of a metallic clip may result in postoperative palpability, potentially causing patient anxiety. This is particularly relevant in Breast Conserving Surgery (BCS), commonly referred to as lumpectomy, where patients are encouraged to perform regular self-examinations following surgery.

Accordingly, there is a need in the industry and field for a device or system that can efficiently and effectively. provide a mapping between a tumor mass and surrounding tissue without being palpable.

### SUMMARY OF THE INVENTION

Therefore, to achieve these and other objects, the herein disclosed invention is surgical specimen margin orientation marking mechanism comprising a plurality of surgical specimen margin markers configured to provide a mapping between a tumor mass and surrounding tissue, wherein the improvement comprises each surgical specimen margin marker comprising sutures placed on tissue to identify one or more locations at which tissue is to be removed or surrounding tissue.

According to the Invention, one or more of said sutures being color coded.

The herein disclosed invention also contemplates A method of marking a tumor mass and surrounding tissue comprising the steps of: positioning a central marking suture to identify a midpoint of said mass; positioning a first set of sutures within said mass at locations around said central marking clip, and positioning a second set of sutures at corresponding locations of surrounding tissue.

Therefore, to achieve these and other objects, in an alternate embodiment, the herein disclosed invention is surgical specimen margin orientation marking mechanism comprising a plurality of surgical specimen margin markers configured to provide a mapping between a tumor mass and surrounding tissue, wherein the improvement comprises each surgical specimen margin marker comprising sutures without clips placed on tissue to identify one or more locations at which tissue is to be removed or surrounding tissue.

### BRIEF DESCRIPTION OF THE DRAWINGS

The present invention will be understood more fully from the detailed description given below and from the accompanying drawings of various embodiments of the invention. The drawings, however, should not be taken to limit the invention to the specific embodiments, but are for explanation and understanding only.

FIGS. 1A & 1B illustrate a conventional surgical specimen margin orientation marking system;

FIGS. 2A and 2B illustrate embodiments of a mass identified by a mapping of surgical specimen margin markers; and

FIG. 3 illustrate another embodiment of surgical specimen margin marker.

### DETAILED DESCRIPTION OF THE INVENTION

An example of surgical specimen margin orientation marking may be featured in an excision of a tumor. In such an example, the tumor is removed and forwarded to a pathologist for evaluation by frozen section or permanent wax-based pathology. The pathologist subsequently performs an analysis to identify malignancy at one or more of the tumor margins. The pathologist may direct the surgeon of additional tissue needing to be excised, the orientation and location of which is critical for proper and accurate excision. Generally, sutures will most often be used in breast cancer surgery—an open surgical procedure.

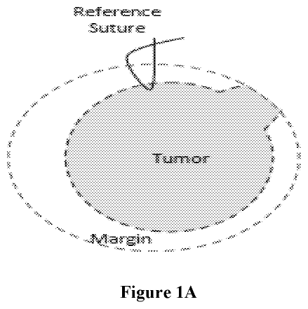

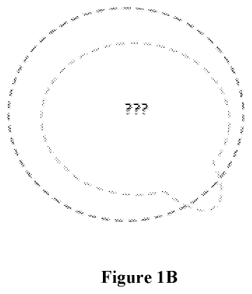

Surgical specimen margin orientation marking typically involves ink marking (e.g., ex-vivo marking) once a specimen has been removed. FIG. 1A illustrates one embodiment of a conventional marking technique in which a specimen mass (or tumor) is marked with a dotted line to differentiate the mass from surrounding tissue (or margin). As shown in FIG. 1A, a reference suture is added at a particular position, such as “12:00 o'clock”, on the mass. As defined herein, ex-vivo marking refers to marking that occurs outside of the body, while in-vivo marking refers to marking that occurs inside of the body.

The specimen is subsequently delivered to a pathologist, who, after examination, communicates to the surgeon locations at which additional tissue is to be removed. However, this approach is inaccurate and subject to error because the technique has no corresponding in-vivo marking, and particularly in instances in which the suture is accidentally removed or the orientation of the specimen is changed (e.g., due to being dropped, manipulated, etc.). For example, the surgeon, staff, or pathologist examining the mass may mistakenly manipulate the removed mass. Even in instances in which the suture has not been removed, there may be miscommunication between surgeons and pathologists related to orientation. As a result, it may be difficult for a surgeon to correctly identify the exact location where additional tissue must be removed, as shown in FIG. 1B.

According to one embodiment, a surgical specimen margin orientation marking mechanism is disclosed that comprises a plurality of surgical specimen margin markers (or specimen markers) configured to provide a mapping between a tumor mass and surrounding tissue. Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

In the following description, numerous details are set forth. It will be apparent, however, to one skilled in the art, that the present invention may be practiced without these specific details. In other instances, well-known structures and devices are shown in block diagram form, rather than in detail, in order to avoid obscuring the present invention.

According to its basic embodiment, the herein disclosed invention is surgical specimen margin orientation marking mechanism comprising a plurality of surgical specimen margin markers configured to provide a mapping between a tumor mass and surrounding tissue, wherein the improvement comprises each surgical specimen margin marker comprising sutures placed on tissue to identify one or more locations at which tissue is to be removed or surrounding tissue.

In a preferred embodiment, one or more of said sutures being color coded.

The herein disclosed invention also contemplates A method of marking a tumor mass and surrounding tissue comprising the steps of: positioning a central marking suture to identify a midpoint of said mass; positioning a first set of sutures within said mass at locations around said central marking clip, and positioning a second set of sutures at corresponding locations of surrounding tissue.

In an alternate embodiment, the herein disclosed invention is surgical specimen margin orientation marking mechanism comprising a plurality of surgical specimen margin markers configured to provide a mapping between a tumor mass and surrounding tissue, wherein the improvement comprises each surgical specimen margin marker comprising sutures without clips placed on tissue to identify one or more locations at which tissue is to be removed or surrounding tissue

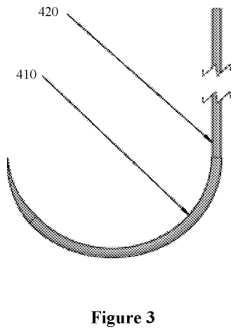

One embodiment is shown in FIG. 3, which illustrates a specimen marker comprising a needle 410 coupled to a suture 420. In this embodiment, the specimen marker comprises a needle 410 coupled to a suture 420. Similar to the marking clip embodiment discussed above, a central needle 410/suture 420 is implemented along with needle 410/suture 420 pairs. In such an embodiment, the central suture 420 and each suture 420 pair comprises a different color (e.g., red, yellow, purple, green and orange).

More, particularly, the surgeon places markers on the tumor and matching markers in the tumor bed—according to the herein disclosed invention, these markers are sutures.

The marked tumor is excised and sent to the pathology lab for examination.

After examination, the Pathologist sees a positive (aka “dirty”) margin and advises the surgeon where tissue must be excised.

The herein disclosed suture markers enable precise communication between the Pathologist and the surgeon. For example, the Pathologist can instruct the surgeon—“Positive margin between the green and purple markers, midline to deep, excise 5 mm.”

In such manner, the surgeon can be timely informed of the cancerous area to be removed. Since the sutures markers are still in place, the surgeon can quickly spot the area to be removed and quickly excise the troublesome tumor.

According to one embodiment of the Invention, surgical specimen margin markers comprise sutures placed on tissue to identify one or more locations at which tissue is to be removed.

According to one embodiment, each suture is color coded to assist in identifying a position of a mass and surrounding tissue. In such an embodiment, color-coded sutures are implemented in pairs to provide a mapping that aligns positions between the mass and the surrounding tissue.

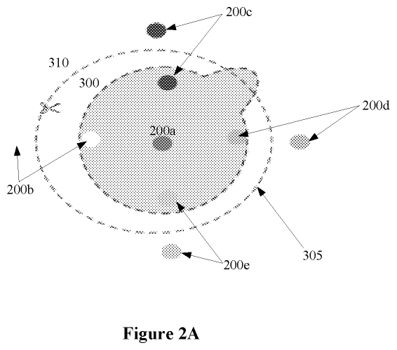

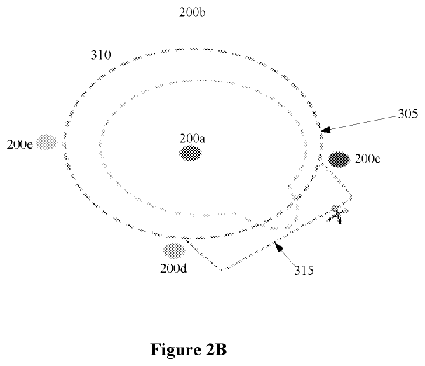

FIGS. 2A & 2B illustrate embodiments of a mass 300 and surrounding tissue 310 identified by a mapping of sutures 200. As shown in FIG. 3A, a mass 300 to be excised is identified by dotted line 305 to differentiate from surrounding tissue 310. In one embodiment, sutures 200 are placed at a plurality of locations of mass 300 and surrounding tissue 310. In this embodiment, a central marking suture 200a is included to identify a midpoint (or midline) of mass 300.

Suture pairs 200b, 200c, 200d and 200e can, in some embodiments, be placed at locations around central marking suture 200a within mass 300 and surrounding tissue 310. For example, sutures 200b, 200c, 200d and 200e are placed within mass 300 at locations around (e.g., 360 degrees) central marking suture 200a. Additionally, a second set of sutures 200b, 200c, 200d and 200e are placed at corresponding locations of surrounding tissue 310. For example, a suture 200b is placed in surrounding tissue 310 at a location adjacent to the location of the suture 200b located within mass 300. Further, a suture 200c is placed in surrounding tissue 310 at a location adjacent to the location of the suture 200c located within mass 300, and so forth with the additional sutures.

According to the Invention, central suture 200a and suture pairs 200b, 200c, 200d and 200e may be comprised of different colors. For instance, suture 200a comprises a first color (e.g., red), while sutures 200b, 200c, 200d and 200e comprise second (e.g., yellow), third (e.g., purple), fourth (e.g., green) and fifth (e.g., orange) colors, respectively. However, other embodiments may feature different color-coding schemes.

Therefore, the Invention thereby allows a surgeon to excise mass 300 along dotted line 305 to be forwarded for pathological examination. Once mass 300 has been removed, suture markers 200b, 200c, 200d and 200e remain inserted in the surrounding tissue while mass 300 is examined. In a further embodiment suture markers 200b, 200c, 200d and 200e remain within the mass 300 are used during examination to provide references. In such an embodiment, the examination of mass 300 may indicate that there is additional tumorous tissue extending into the surrounding tissue 310. Accordingly, examination instructions may be provided with reference to the suture markers 200. For example, instructions may be provided to remove a portion of additional surrounding tissue 310 between suture markers 200c and 200d. Subsequently, the surgeon uses suture markers 200c and 200d as references to remove the additional surrounding tissue 310 along dotted line 315, as shown in FIG. 3B.

The above-described embodiments, provides a plurality of reference points at locations within a tumor mass, as well as matching points in surrounding tissue to create a mapping that enables precise communications between a surgeon and pathologist.

Whereas many alterations and modifications of the present invention will no doubt become apparent to a person of ordinary skill in the art after having read the foregoing description, it is to be understood that any particular embodiment shown and described by way of illustration is in no way intended to be considered limiting. Therefore, references to details of various embodiments are not intended to limit the scope of the claims, which in themselves recite only those features regarded as the invention.

I claim:

1. A surgical specimen margin orientation marking mechanism comprises a plurality of surgical specimen margin markers configured to provide a mapping between a tumor mass and surrounding tissue, wherein the improvement comprises each surgical specimen margin marker comprising sutures placed on tissue to identify one or more locations at which tissue is to be removed or surrounding tissue.

2. A mechanism according to claim 1, wherein one or more of said sutures being color coded.

3. A method of marking a tumor mass and surrounding tissue comprising: positioning a central marking suture to identify a midpoint of said mass; positioning a first set of sutures within said mass at locations around said central marking clip, and positioning a second set of sutures at corresponding locations of surrounding tissue

4. A surgical specimen margin orientation marking mechanism comprises a plurality of surgical specimen margin markers configured to provide a mapping between a tumor mass and surrounding tissue, wherein the improvement comprises each surgical specimen margin marker comprising sutures without clips placed on tissue to identify one or more locations at which tissue is to be removed or surrounding tissue.
